(19) United States
(12) Patent Application Publication
(10) Pub. No.: US 2017/0262588 A1
(43) Pub. Date: Sep. 14, 2017

(54) CHILD HEALTH MANAGEMENT SYSTEM AND CHILD HEALTH MANAGEMENT METHOD

(71) Applicant: MOBILE DOCTOR CO.,LTD., Seoul (KR)

(72) Inventor: Jae Won SHIN, Gwangmyeong-si (KR)

(21) Appl. No.: 15/324,771

(22) PCT Filed: Jul. 9, 2015
(86) PCT No.: PCT/KR2015/007155

§ 371 Date: Jan. 9, 2017

(30) Foreign Application Priority Data
Jul. 10, 2014 (KR) 10-2014-0087023

(51) Int. Cl. G06F 19/00; A61B 5/00

(52) U.S. Cl. CPC G06F 19/325; G06F 19/322; A61B 5/0008

(57) ABSTRACT

Provided are a child health management system and a child health management method. A child health management system according to one embodiment of the present invention is a child health management system using a user terminal, which comprises: a storage unit for storing condition information of a child and surrounding environment information of the child, transmitted from the user terminal; a health condition analysis unit for analyzing the health condition of the child on the basis of the condition information and surrounding environment information of the child, stored in the storage unit; and a response strategy deduction unit for deducing a user's response strategy according to the analysis result from the health condition analysis unit.

### CROSS-REFERENCE TO RELATED APPLICATIONS

This patent application is a national-stage application of International Patent Application No. PCT/KR2015/007155 filed on Jul. 9, 2015 which claims priority under 35 U.S.C. §119 to Korean Patent Application No. 10-2014-0087023, filed on Jul. 10, 2014, in the Korean Intellectual Property Office, the disclosures of which are incorporated by reference herein in their entireties.

### TECHNICAL FIELD

The present invention relates to child health-care systems and methods, and more specifically, to child health-care systems and methods capable of analyzing health conditions of a child based on information about the child and providing a treatment.

### DISCUSSION OF RELATED ART

A patient who feels uncomfortable may immediately visit a doctor's office to receive a medical service during the daytime on weekdays or in the t morning on Saturday. At nighttime on weekdays or on weekends, however, every hospital, except its emergency room, closes. A patient may, of course, receive a medical treatment from the emergency room, but he ma not, due to a limited number of emergency rooms, lack of close urgent care facilities or otherwise in certain circumstances. Moreover, he might not be under a clear determination as to whether he must rush to the emergency room as in the case where he simply suffers from slight fever and its resultant pain.

In such case, they tend to rely on public medical books or medical knowledge shared on personal blogs or webpages, or social networking services (SNSs) or other online communities for a proper treatment. They oftentimes use easily accessible medical instruments as necessary.

### SUMMARY

A treatment based on public medical knowledge would not impose a significant harm on the health of an adult patient who can make a judgement on his own body condition and has bodily immunity.

However, child patients, particularly infants or toddlers uncappable of clear communication, may not determine and deliver their body condition, nor can their parent, a non-expert in medical fields, make an adequate determination thereon. Further, the parent's treatment based on a misjudgment would worsen the patient's condition because children have less immunity as compared with grown-ups. For example, treating with a fever reliever should be preceded by a determination as to what type or dosage of it should be given, which is not easy to make by parents in an appropriate manner.

The present invention has been conceived to address the above issues, and an object of the present invention is to provide a child health-care system and method capable of analyzing a child's health condition based on information about the child and providing a treatment depending on a result of the analysis.

Another object of the present invention is to provide a child health-care system and method capable of determination with high accuracy by further considering various factors, e.g., ambient environmental information about the child, as well as condition information about the child when analyzing the child's health condition and producing a treatment depending thereon.

Still another object of the present invention is to provide a child health-care system and method that steadily receives and stores condition information and ambient environmental information about a child and supplies the stored information to a medical expert, allowing the medical expert to make a right medical judgement based on sufficient information when the child receives a consultation from the medical expert through the system or receives a checkup in the hospital in the future.

Objects of the present invention are not limited to the foregoing, and other unmentioned objects would also be apparent to one of ordinary skill in the art from the following description.

According to the present invention, a child's health condition may be analyzed based on information about the child, and a treatment may be provided depending on a result of the analysis.

Further, according to the present invention, various factors, e.g., ambient environmental information about a child, as well as condition information about the child are considered when analyzing the child's health condition and producing a treatment depending thereto, thereby increasing the accuracy of determination.

Further, according to the present invention, condition information about a child and ambient environmental information about the child are steadily received and stored, and the stored information is supplied to a medical expert, allowing the medical expert to make a right medical judgement based on sufficient information when the child receives a consultation from the medical expert through the system or receives a checkup in the hospital in the future.

### BRIEF DESCRIPTION OF THE DRAWINGS

FIG. 1 is a view schematically illustrating a use environment of a child health-care system according to an embodiment of the present invention;

FIG. 2 is a view schematically illustrating a child health-care system according to an embodiment of the present invention;

FIG. 3 is a view schematically illustrating a configuration of a storage unit as illustrated in FIG. 2;

FIG. 4 is a view illustrating transmissions and receptions of a child health-care system according to an embodiment of the present invention;

FIG. 5 is a flowchart illustrating a child health-care method according to an embodiment of the present invention;

FIG. 6 is a flowchart illustrating a child health-care method according to another embodiment of the present invention; and

FIG. 7 is a flowchart illustrating a child health-care method according to still another embodiment of the present invention.

### DESCRIPTION OF EXEMPLARY EMBODIMENTS

Hereinafter, preferred embodiments of the present invention are described in detail with reference to the accompanying drawings. Advantages and features of the present invention and methods for achieving the same will be apparent from the detailed description of the following embodiments of the present invention taken in conjunction with the drawings. However, the present invention may be embodied in other various ways without being limited to the embodiments set forth below which are provided simply for the purpose of rendering the present invention disclosed thoroughly while letting the category of the present invention known to one of ordinary skill in the art. The present invention is defined only by the scope of the claims. The same reference denotations are used to refer to the same elements throughout the specification.

Unless defined otherwise, all of the terms, technical or scientific, as used herein may be used in the meanings commonly understood by one of ordinary skill in the art. Terms defined in a dictionary generally in use should not be construed ideally or overly unless defined particularly.

The terms as used herein are provided for describing embodiments, but not intended to limit the present invention. In the disclosure, a singular form includes a plural form unless mentioned specifically. The terms “comprise(s)” and/or “comprising” as used herein do not exclude the presence or addition of one or more components other than the mentioned component(s). Further, as used herein, the term “unit” may be configured in hardware or software, but not limited thereto.

Now described are a child health-care system and method according to embodiments of the present invention with reference to the drawings.

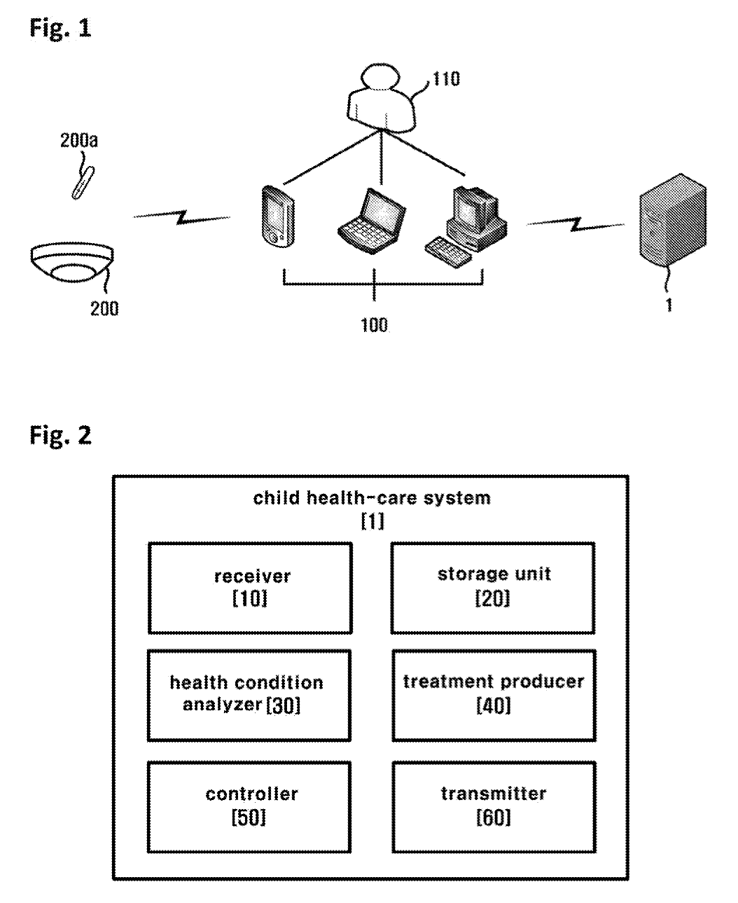

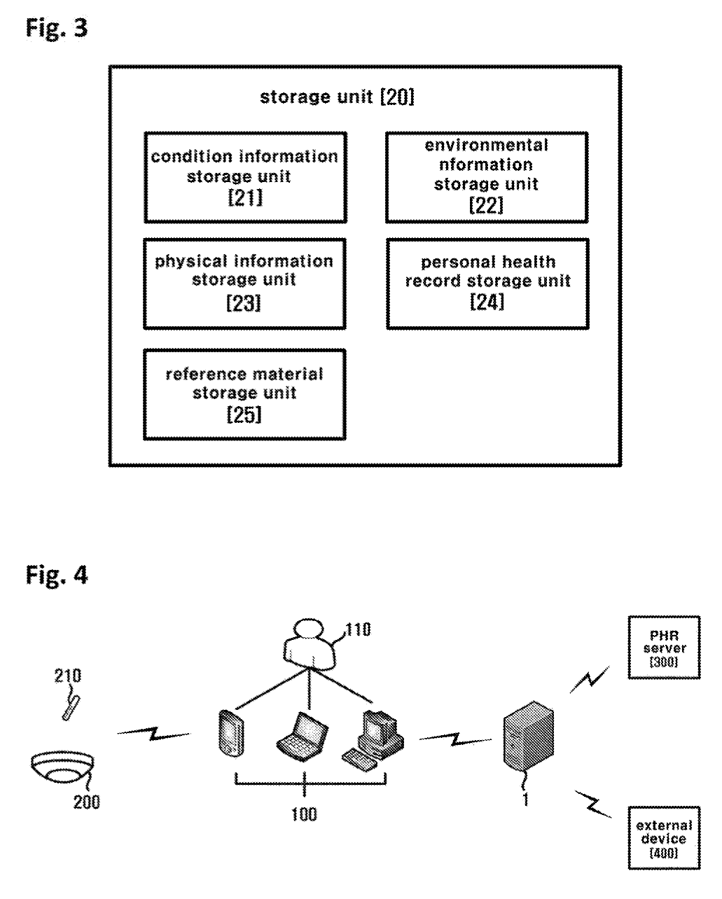

Referring to FIGS. 1 to 4, according to an embodiment of the present invention, a child health-care system 1 is described. FIG. 1 is a view schematically illustrating a use environment of a child health-care system 1 according to an embodiment of the present invention. FIG. 2 is a view schematically illustrating a child health-care system 1 according to an embodiment of the present invention. FIG. 3 is a view schematically illustrating a configuration of a storage unit 20 as illustrated in FIG. 2. FIG. 4 is a view illustrating transmissions and receptions of a child health-care system 1 according to an embodiment of the present invention.

Referring to FIG. 1, according to an embodiment of the present invention, the child health-care system 1 may function as a database and server, may use a user terminal 100, and may connect with the user terminal 100 via a network. In other words, the child health-care system 1 may receive information about a child, as a target of monitoring, from the user terminal 100, may analyze the received information and analyze the child's health condition, and may produce a user's treatment according to a result of the analysis, and provide the treatment to the user terminal 100. Various types of wired or wireless communication schemes may be used for communication between the child health-care system 1 and the user terminal 100, without limitation on the network used.

The user terminal 100 may be an electronic device that may exchange information with the child health-care system 1, e.g., including, but not limited to, a mobile phone, a cellular phone, a smartphone, a personal digital assistant (PDA), a portable multi-media player (PMP), a tablet PC, a personal computer (PC), a laptop computer, or a wireless communication device, For connection with the child health-care system 1, the user terminal 1 may have installed a dedicated program (or a dedicated application) for the child health-care system 1, but without limited thereto, the user terminal 100 may connect to the child health-care system 1 via the web.

To obtain information about the child, the user terminal 100 may interwork with various external sensor(s) 200 and may receive information sensed by the external sensor(s) 200. For example, when the user terminal 100 interworks with a thermometer 200a, the thermometer 200a may read body temperature information on the child or a parent of the child and transmit the read body temperature information to the child health-care system 1 through the network. However, a device interworkable with the user terminal 100 is not limited to the thermometer 200a or other external sensors 200, and any device capable of providing condition information about the child or ambient environmental information about the child may interwork with the user terminal 100 by the dedicated program (or dedicated application). Accordingly, the child health-care system 1 may gather various information using the user terminal 100.

Other than receiving information sensed by the external sensor 200 interworking with the user terminal 100, the user terminal 100 may directly receive information from a user 110, in other words, the user 110 may enter, e.g., condition information about the child, ambient environmental information about the child, or physical information about the child to the user terminal 100. For example, the user 100 may read the child's body, temperature using the thermometer 200a and may then directly enter the read body temperature information to the user terminal 100, or the user 110 may observe the child's symptom and enter symptom information about the child to the user terminal 100. Thus, the condition information about the child or ambient environmental information about the child may directly be entered to the user terminal 100 by the user 110 or may be transmitted from the external sensor 200 interworking with the user terminal 100 to the user terminal 100 and then transmitted from the user terminal 100 to the child health-care system 1.

Meanwhile, as used herein, the user 110 may be a guardian taking care of the child, as well as the child's parent, but is not limited thereto. The user terminal 100 may transmit information received from an interworking external sensor 200 or the user 110 to the child health-care system 1 through the network.

A child health-care system 1 according to an embodiment of the present invention is described below with reference to FIG. 2.

Referring to FIG. 2, the child health-care system 1 may include a receiver 10, a storage unit 20, a health condition analyzer 30, a treatment producer 40, a controller 50, and a transmitter 60. However, the components shown in FIG. 2 are not essential elements, and the child health-care system 1 may be implemented to include more or less components.

The receiver 10 may receive information from the user terminal 100. For example, the receiver 10 may receive the condition information about the child and the ambient environmental information about the child from the user terminal 100. However, information received by the receiver 10 from the user terminal 100 is not limited thereto, and as necessary, login information may be received from the user terminal 100. The receiver 10 may also receive information from a personal health record (PHR) server 300 or an external device 400 as described below.

The receiver 10 may be, e.g., a sort of communication module for receiving data but is not limited thereto.

The storage unit 20 may store the condition information about the child and the ambient environmental information about the child transmitted from the user terminal 100. The storage unit 20 may further store physical information about the child transmitted from the user terminal 100, the child's personal health record (PHR) transmitted from the PHR server 300, and a reference material that may be referred to upon analyzing the child's health condition and producing a treatment. However, information stored in the storage unit 20 is not limited thereto.

Referring to FIG. 3, the storage unit 20 may include a condition information storage unit 21, an environmental information storage unit 22, a personal health record storage unit 24, a physical information storage unit 23, and a reference material storage unit 25. However, the components shown in FIG. 3 are not inevitable, and the storage unit 20 may include more or less components.

The condition information storage unit 21 may store condition information about the child transmitted from the user terminal 100. The condition information about the child may be information about the child's current condition or state and may include, e.g., body temperature information about the child and symptom information about the child. The symptom information about the child may be information regarding a symptom observed from the child that is monitored, such as vomit, diarrhea, or cough. However, the condition information about the child is not limited thereto and may also include information about the administration or urgent care performed on the child by the user.

According to an embodiment of the present invention, since the child health-care system 1 stores information about the child's symptom as well as the body temperature information about the child, the system 1 may analyze the child's health condition based on sufficient information and produce a treatment as compared with when performing analysis on the child's health condition simply considering the body temperature information about the child. Therefore, information provided by the child health-care system 1 may be more reliable.

Further, according to an embodiment of the present invention, the child health-care system 1 may steadily store the body temperature information about the child, which enables generation of a body temperature chart in which variations in the body temperature are recorded over time.

The environmental information storage unit 22 may store information about an ambient environment of the child transmitted from the user terminal 100. The ambient environmental information may be information about a factor that may have an influence on the child's health condition and may include, e.g., body temperature information about at least one of the child's parents and may also include, e.g., an ambient temperature, humidity, noise level, time, or data, but is not limited thereto.

In the child health-care system 1 according to an embodiment of the present invention, since the ambient environmental information about the child is stored by the environmental information storage unit 22, information about what environment the child has been exposed may be obtained. Therefore, a stereoscopic analysis is rendered possible given various factors affecting the child's health condition. Further, when the user 110 proceeds with a consultation with a medical expert through the child health-care system 1, the ambient environmental information about the child may be provided to the medical expert, allowing the medical expert to examine the child's health condition through objective information.

For example, in the child health-care system 1 according to an embodiment of the present invention, the storage unit 20 has body temperature information about the child's parent(s) as well as the body temperature information about the child. Body temperature may be subject to a deviation depending on the characteristics of a thermometer in use. Thus, retaining only body temperature information about the child may fail to provide a determination as to the characteristics of the thermometer or whether it has an error. However, according to this embodiment, because the body temperature information about the child's parent(s) is stored as well, an objective determination may be made on the reliability of the thermometer by a relative comparison with the body temperature information about the parent(s).

The physical information storage unit 23 may store physical information about the child transmitted from the user terminal 100. The physical information about the child may include, e.g., age (or the number of months), height, weight, or other body information regarding the child.

According to an embodiment of the present invention, in the child health-care system 1, since the storage unit 20 stores the physical information about the child, analysis of the health condition or production of a treatment according to the physical information about the child may be rendered possible. For example, given the fact that the child has a different physical property depending on his age (or number of months), a dosage of medication may be adjusted.

Referring to FIG. 4, the personal health record storage unit 24 may store a personal health record (PHR) transmitted from the PHR server 300. A PHR may be a record containing all health information about a person and may include information about the person's disease or treatment history. The PHR may be stored and managed by the PHR server 300. The child health-care system may receive the child's PHR from the PHR server 300 and store the received PHR. Accordingly, according to an embodiment of the present invention, the child health-care system 1 may obtain information about the child's past disease history through the PHR and may thus refer to the same to determine factors that should be taken into accordance upon analysis of the child's health condition or may also refer to the PHR when producing a treatment.

The reference material storage unit 25 may store a reference material. The reference material may be referred to when the health condition analyzer 30 analyzes the child' health condition or the treatment producer 40 produces a treatment according to the child's health condition as described below. For example, the reference material may be one in which the child's health conditions corresponding to the child's body temperatures and symptoms are stored in the form of a lookup table or one in which the user's treatments corresponding to the child's health conditions are stored in the form of a lookup table, but is not limited thereto or thereby.

The health condition analyzer 30 may analyze the child's health condition based on the condition information, and ambient environmental information about the child stored in the storage unit 20. When the storage unit 20 further stores the physical information about the child and the child's PHR, the health condition analyzer 30 may analyze the child's health condition also considering the physical information about the child and the child's PHR.

Specifically, the health condition analyzer 30 may process the information stored. in the storage unit 20 to analyze the child's health condition and may analyze the child's health condition using the processed information. Processing the stored information may include processing or arranging the stored pieces of information for easier analysis on the child's health condition. For example, the health condition analyzer 30 may detect a per-time period variation in the child's body temperature information, there by grasping how much the child's body temperature changes and a trend in the variation. Further, in some embodiments, the health condition analyzer 30 may consider the body temperature information about a parent(s) or an ambient factor, such as an ambient temperature or humidity, as well as the body temperature information about the child.

The analysis of the child's health condition is to determine whether the child has a health abnormality using the stored or processed information, enabling elicitation of a disease of suspect or its name or determination as to whether it is urgent. The reference material may come into use for the analysis of the child's health condition. For example, the health condition analyzer 30 may elicit the child's health condition corresponding to the stored or processed information.

In the child health-care system 1, according to an embodiment of the present invention, not simply the body temperature information about the child but also the ambient environmental information is put to use, thus taking into consideration what circumstance the child is in exposure to. The child's age, height, or weight may be considered through the physical information about the child, and the child's past disease history may be accounted for through the child's personal health record. As such, the child health-care system 1, according to an embodiment of the present invention, may perform an objective analysis on the child's health condition by considering various, not only one, possible factors upon doing so.

The treatment producer 40 may produce the user's treatment according to a result of the analysis of the child's health condition by the, health condition analyzer 30. The user's treatment produced from the treatment producer 40 may be one that may be performed by the user 110 taking care of the child—e.g., administering a medication not requiring a medical doctor's prescription, such as a fever reliever, or urgent care that the user may exercise on the child, such as applying an ice bag.

A specific example of the administration of a fever reliever is described. The treatment producer 40 may specifically provide information about how much fever reliever should be administered considering the physical information about the child, information about which type of fever reliever among a liquid medicine, a chewable tablet, and a suppository is preferable for the child, and other information about, e.g., a frequency of taking the fever reliever, in other words, the child health-care system 1, according to an embodiment of the present invention, may offer the user 110 a specific treatment as well as a brief guide, allowing the user 110 to exercise the treatment with little difficulty.

Further, the treatment producer 40 may also produce a scheme for the user to visit an emergency room. In cases where a result of the analysis by the health condition analyzer 30 reveals that the child is in emergency requiring a precise checkup or treatment by a medical expert, the treatment producer 40 may presents a suggestion that the user visit an emergency room as a treatment. In addition, the treatment producer 40 may also provide information about an emergency room close to where the user terminal 100 is located.

Meanwhile, referring to FIG. 4, where the user 110 visits an emergency room, if there is a request for information about the child from the user terminal 100, the controller 50 may provide the information about the child stored in the storage unit 20 to the user terminal 100 or an external device 400 associated with the emergency room, such as an external terminal or external server designated by the user 110.

Meanwhile, the result of analysis by the health condition analyzer 30 and the treatment produced by the treatment producer 40 may be transmitted from the transmitter 60 to the user terminal 100 under the control of the controller 50, and the user 110 may identify such information through a display or voice.

The controller 50 may control each component, including the storage unit 20, in the child health-care system 1. For example, the controller 50, if there is a request for the information about the child stored in the storage unit 20 from the user terminal 100, may transmit the information stored in the storage unit 20 through the transmitter 60 to the user terminal 100. For example, where there is a request for body temperature information about the child, the controller 50 may send the stored body temperature information about the child through the transmitter 60 to the user terminal 100, and thus, the body temperature information about the child, which varies over time, may be displayed on the user terminal 100.

According to a request from the user terminal 100, the controller 50 may send the information about the child stored in the storage unit 20 through the user terminal 100 to the designated external device 400, thus allowing for an electronic medical record (EMR) interoperation with the hospital and resultantly supplying a medical doctor with sufficient information for examination.

Further, where there is a request for a consultation service with the medical expert from the user terminal 100, the controller 50 may connect the external device 400, which is associated with the medical expert, to the user terminal 100. Accordingly, the user 110 may proceed with consultation with the medical expert through the user terminal 100. As necessary, the controller 50 may provide the information about the child stored in the storage unit 20 to the external device associated with the medical expert, allowing the medical expert to counsel with sufficient information.

In the meantime, the controller 50 may send a notification to the user terminal 100 through the transmitter 60 so that information about the child, including body temperature information about the child, may be measured every predetermined time. Thus, the user terminal 100 may request the external sensor 200 to send measured information or the user 110 to enter information.

The transmitter 60 may be a sort of communication module for transmission of, e.g., data, and the transmitter 60 may transmit information stored in the child health-care system 1 to the external device under the control of the controller 50.

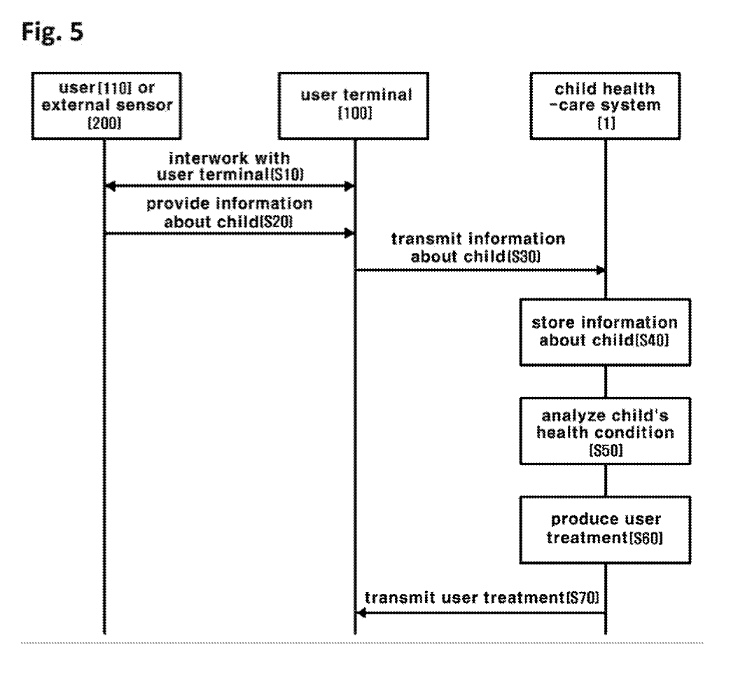

Subsequently, referring to FIG. 5, a child health-care method according to an embodiment of the present invention is described. FIG. 5 is a flowchart illustrating a child health-care method according to an embodiment of the present invention. Since the technical features of the child health-care system 1 according to an embodiment of the present invention may be applicable to the child health-care method according to an embodiment of the present invention, no repetitive description is given.

First, referring to FIG. 5, a user terminal 100 may interwork with an external sensor 200 to sense condition information about a child or ambient environmental information about the child (S10). No limitation is imposed on the external sensor 200 interworking with the user terminal 100, and the user terminal 100 may receive various information through the interworking.

Then, a user 110 may provide the information about the child to the user terminal 100 by direct entry, and the external sensor 200 may provide sensed information to the user terminal 100 through a network (S20).

Next, the user terminal 100 may transmit the information about the child provided from the user 110 or the external sensor 200 to the child health-care system 1 (S30).

Subsequently, the child health-care system 1 may store condition information about the child and ambient environmental information about the child transmitted from the user terminal 100 (S40). Physical information about the child transmitted from the user terminal 100 may also be stored in the child health-care system 1, and as necessary, the child's personal health record transmitted from a personal health record (PHR) server 300 may also be stored in the child health-care system 1.

The child health-care system 1 may then analyze the child's health condition based on the stored condition information about the child and ambient environmental information about the child (S50). If necessary, the physical information about the child or personal health record may be used for the analysis of the child's health condition.

Then, the child health-care system 1 may produce the user's treatment according to a result of the analysis of the child's health condition (S60).

Subsequently, the user's treatment may be transmitted from the child health-care system 1 to the user terminal 100 (S70). However, as necessary, the result of analysis of the child's health condition may also be provided from the child health-care system 1 to the user terminal 100.

Therefore, the user 110 may utilize the treatment transmitted to the user terminal 100.

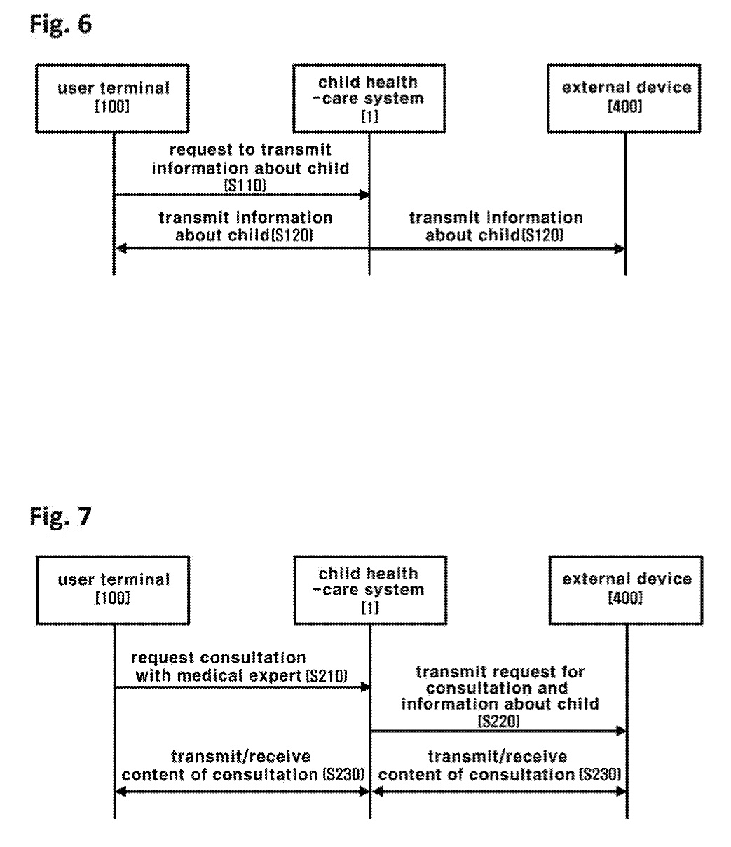

Now described with reference to FIG. 6 is a child health-care method according to another embodiment of the present invention. FIG. 6 is a flowchart illustrating a child health-care method according to another embodiment of the present invention. The description primarily focuses on differences from the child health-care method set forth above.

Referring to FIG. 6, a user terminal 100 may request the child health-care system 1 to transmit information about a child to the user terminal 100 or a designated external device 400.

Accordingly, the child health-care system 1 may transmit stored information about the child to the user terminal 100 or the designated external device 400 (S120).

A child health-care method according to still another embodiment of the present invention is described below with reference to FIG. 7. FIG. 7 is a flowchart illustrating a child health-care method according to still another embodiment of the present invention. The description mainly focuses on differences from the child health-care method described supra.

Referring to FIG. 7, a user terminal 100 may send a request for consultation with a medical expert to the child health-care system 1 (S210).

The child health-care system 1 may then send a request for consultation to an external device 400 associated with a designated medical expert and transmit stored information about a child subject to the consultation (S220).

Accordingly, the child health-care system 1 may transmit and receive the contents of the consultation between the user terminal 100 and the external device 400 (S230), so that the user 110 may receive a consultation as to the child's health condition and treatment from the medical expert through the child health-care system 1.

Although embodiments of the present invention have been described with reference to the accompanying drawings, it should be appreciated by one of ordinary skill in the art that the present invention may be embodied in other various forms without departing from the technical spirit or essential features thereof. Therefore, the embodiments set forth above should be construed as exemplary but rather than limiting in all aspects.

What is claimed is:

1. A child health-care system using a user terminal, comprising:
a storage unit storing condition information about a child and ambient environmental information about the child transmitted from the user terminal;
a health condition analyzer analyzing the child's health condition based on the condition information about the child and the ambient environmental information about the child stored in the storage unit; and
a treatment producer producing a user's treatment according to a result of the analysis v the health condition analyzer.

2. The child health-care system of claim 1, wherein the condition information about the child includes body temperature information about the child and symptom information about the child.

3. The child health-care system of claim 2, wherein the ambient environmental information about the child includes body temperature information about at least one of the child's parents.

4. The child health-care system of claim 1, wherein the storage unit further stores physical information about the child transmitted from the user terminal, and wherein the health condition analyzer analyzes the child's health condition based on the stored condition information about the child, ambient environmental information about the child, and physical information about the child.

5. The child health-care system of claim 1, wherein the storage unit further stores a personal health record (PHR) of the child transmitted from an external PHR server, and wherein the health condition, analyzer analyzes the child's health condition based on the stored condition information about the child, ambient environmental information about the child, and the child's PHR.

6. The child health-care system of claim 1, wherein producing the user's treatment according to the result of the analysis is producing at least one of an administration scheme, an emergency care scheme, and a visit scheme according to the result of the analysis.

7. The child health-care system of claim 1, wherein the condition information about the child and the ambient environmental information about the child are directly entered to the user terminal by the user or are transmitted from an external sensor interworking with the user terminal to the user terminal and then transmitted from the user terminal to the child health-care system.

8. The child health-care system of claim 1, further comprising a controller controlling the storage unit, wherein the controller performs control so that the condition information about the child and the ambient environmental information about the child stored in the storage unit are transmitted to an external device according to a request from the user terminal.

9. A child health-care method using a user terminal, comprising:
storing condition information about a child and ambient environmental information about the child transmitted from the user terminal;
analyzing the child's health condition based on the stored condition information about the child and the ambient environmental information about the child; and
producing a user's treatment according to a result of the analysis of the child's health condition.

10. The child health-care method of claim 9, wherein the condition information about the child includes body temperature information about the child and symptom information about the child.
